(19) United States
(12) Patent Application Publication
(10) Pub. No.: US 2015/0133701 A1
(43) Pub. Date: May 14, 2015

(54) Integrated Steam Methane Reformer and Hydrogenation of Acetic Acid to Produce Ethanol

(71) Applicant: Celanese International Corporation, Irving, TX (US)

(72) Inventors: David Townsend, Friendswood, TX (US); Andrew Shuff, League City, TX (US)

(73) Assignee: Celanese International Corporation, Irving, TX (US)

(21) Appl. No.: 14/075,182

(22) Filed: Nov. 8, 2013

(51) Int. Cl. C07C 29/149

(52) U.S. Cl. CPC C07C 29/149
USPC 568/885

(57) ABSTRACT

A process is disclosed for integrating a steam methane reformer to produce hydrogen that is used for converting acetic acid and/or ethyl acetate to ethanol. The process may use a methane-containing stream obtained from a stranded natural gas or associated gas source. The process water from the hydrogenation reaction is used to saturate the methane-containing stream. The process water comprises water and oxygenates, wherein the maximum amount of oxygenates is less than or equal to 10 wt. %. Processes for integrating fuel sources, steam and electricity are also disclosed.

### FIELD OF THE INVENTION

The present invention relates generally to integrated processes, systems, and apparatuses for producing ethanol. More particularly, the invention relates to systems, methods and apparatuses that use stranded natural gas and/or associated gas as a hydrogen feedstock for hydrogenating acids and esters thereof to ethanol.

### BACKGROUND OF THE INVENTION

Stranded natural gas is a remote reserve of natural gas that is unusable due to numerous physically or economically problems. The infrastructure to transport the stranded natural may be cost prohibitive to its location. Associated gas, e.g., natural gas found within an oil well, is also unusable and is generally flared. Flaring represents a loss in economic value and a waste that creates environmental concerns. Although estimates vary, roughly half of natural gas reserves may be considered as stranded natural gas or associated gas. Some methods currently considered to commercialize stranded natural gas are liquefaction, conversion to a liquid (syncrude), or compression of natural gas. Other processes have sought to improve the transportation from remote locations to suitable markets. Still other processes have sought to use stranded natural gas for producing different industrial chemicals such as urea, as described in US Pub. No. 2012/0136172, and higher hydrocarbons, as shown in U.S. Pat. No. 7,829,602.

EP0167300 discloses a process for the production of an aliphatic alcohol having at least two carbon atoms, preferably ethanol, from a carbonaceous feedstock, preferably natural gas, via an intermediate aliphatic alcohol having one less carbon atom, preferably methanol, via an intermediate compound containing the group CH₃(CH₂)ₙC(O)—, preferably acetic acid. The feedstock is reformed and the synthesis gas formed is separated, preferably by a PSA unit, into three different streams which are used in the three stage process, one of which streams is a pure hydrogen stream which can be used for the concurrent production of ammonia. The ethanol formed is useful as a petrol extender and octane improver for automobile fuel.

EP2060553 describes a process for converting hydrocarbons to ethanol involving converting the hydrocarbons to ethanoic acid and hydrogenating the ethanoic acid to ethanol. The stream from the hydrogenation reactor is separated to obtain an ethanol stream and a stream of acetic acid and ethyl acetate, which is recycled to the hydrogenation reactor.

The need remains for improving the economic recovery from stranded natural gas sources and associated gas by achieving efficiencies with integration production of industrial chemicals.

### SUMMARY OF THE INVENTION

In a first embodiment, the present invention is directed to a process for producing ethanol comprising hydrogenating acetic acid in a first reactor in the presence of a first catalyst to produce a crude ethanol stream; separating the crude ethanol stream into an ethanol product stream and a process stream comprising water and oxygenates, wherein the process stream comprises less than or equal to 10 wt. % oxygenates; mixing the process stream and a methane-containing stream to produce a feed stream; reforming the feed stream in a second reactor in the presence of a second catalyst to produce a reformed effluent comprising hydrogen, carbon monoxide and carbon dioxide; shifting substantially of all of the carbon monoxide in the reformed effluent to carbon dioxide and hydrogen; and removing substantially of all of the carbon dioxide to yield a pure hydrogen stream that is fed to the first reactor. In one embodiment, the mixing the process stream and a methane-containing stream, the water in the process stream, and optionally the oxygenates, may be vaporized.

In a second embodiment, the present invention is directed to a process for producing ethanol comprising: hydrogenating acetic acid in a first reactor in the presence of a catalyst to produce a vapor crude ethanol stream; condensing the vapor crude ethanol stream to obtain a liquid stream and a hydrogen recycle stream; purging a gaseous portion of the hydrogen recycle stream; separating the liquid stream into an ethanol product stream and a process stream comprising water and oxygenates, wherein the process stream comprises less than or equal to 10 wt. % oxygenates; mixing the process stream and a methane-containing stream to produce a feed stream; reforming the feed stream in a second reactor in the presence of a second catalyst to yield a pure hydrogen stream that is fed to the first reactor, wherein the second reactor comprises a convection section; and introducing the purged gaseous portion to the convection section. The purged gas may be combusted in the convection section to provide heat for the second reactor, i.e., reformer. In one embodiment, the convection section may comprise a furnace and an auxiliary firing section.

### BRIEF DESCRIPTION OF DRAWINGS

The invention is described in detail below with reference to the appended drawings, wherein like numerals designate similar parts.

FIG. 1 is a general flow chart an integrated hydrogen production and ethanol production system in accordance with one embodiment of the present invention.

FIG. 2 is a detailed schematic of a process stream integrated with a hydrogen production in accordance with one embodiment of the present invention.

FIG. 3 is a detailed schematic of an integrating exhaust from a turbine to compress a hydrogen recycle in the ethanol production with a furnace for the steam reforming in accordance with one embodiment of the present invention.

### DETAILED DESCRIPTION OF THE INVENTION

### Introduction

The present invention relates to integration of a hydrogen production unit and an ethanol production unit that improves energy efficiencies and reduces wastewater discharge. Advantageously, reducing wastewater discharge may reduce the need to purify water streams from the ethanol production unit and thus leads to additional improvements in capital and energy efficiencies. The integration efficiencies achieved by the present invention allow for capturing value from stranded natural gas or associated gas. As indicated above, stranded natural gas or associated gas is often in remote locations that make recovery and delivery to markets cost prohibitive. Using embodiments of the present invention, stranded natural gas is converted to hydrogen which is used to produce ethanol that may be transported from the remote location to commercial markets.

Ethanol production units generate a significant amount of wastewater discharge. Although this may be cleaned prior to discharge, the associated costs make this prohibitive. Due to the presence of organics in the wastewater discharge there may additional difficulties in cleaning the wastewater discharge. The present invention provides an advantageous integration that uses the process stream, from the ethanol production in the hydrogen production unit. The process stream may comprise water and less than or equal to 10 wt. % oxygenates, e.g. from 0.001 to 10 wt. % oxygenates.

When producing a hydrogen feedstock from steam methane reforming, a large volume of water is needed. The necessary volume of water may be difficult to obtain depending on the geographical location of the stranded natural gas supply. To overcome this limitation on exploiting stranded natural gas, in one embodiment the present invention integrates a process stream comprising water and oxygenates, with the steam methane reforming, wherein the process stream comprises less than or equal to 10 wt. % oxygenates. The ethanol production generates the necessary volume of water that can be readily used in the steam methane reforming. This reduces the need to use external water, which may be difficult to locate with the stranded natural gas supplies.

The composition of the process stream may vary depending on the hydrogenation reaction conditions used to form ethanol, and on the recovery of ethanol. To maximize the water integration and minimize wastewater discharge, at least 70% of the water from the crude ethanol product, i.e. water produced in the hydrogenation reaction, is recovered in the process stream. More preferably at least 90%, e.g., at least 95% of the water is recovered in the process stream. As water is recovered, one or more oxygenates may be concentrated in the process stream. Additional purification may be needed to remove these oxygenates if the water is purged and discharged from the ethanol production unit. However, the present invention avoids the additional purification, and thus avoids the associated capital and energy costs, by withdrawing a process stream that may be readily integrated with a hydrogen production unit. In one embodiment, the process stream comprises water and oxygenates, e.g., the process stream comprises less than or equal to 10 wt. % oxygenates, e.g., less than 5 wt. % oxygenates or less than 1 wt. % oxygenates. In terms of ranges, the process stream may comprise from 90 wt. % to 99.999 wt. % water and 0.001 wt. % to 10 wt. % oxygenates, e.g., from 95 wt. % to 99.9 wt. % water and 0.1 wt. % to 5 wt. % oxygenates. Oxygenates may include acetic acid, ethyl acetate, acetaldehyde, diethyl acetal, diethyl ether, and optionally alcohols such as ethanol, n-propanol, isopropanol, n-butanol, isobutanol. Because hydrocarbons, mainly methane and ethane, do not concentrate with water during the ethanol recovery, the process stream typically does not comprise hydrocarbons. Preferably oxygenates comprise acetic acid and may comprise one other oxygenate in a lesser amount than the acetic acid. In one exemplary embodiment, the process stream comprises at least 90 wt. % water and less than 10 wt. % acetic acid, e.g., less than 5 wt. % acetic acid or less than 1 wt. % acetic acid. The other oxygenate compounds, including alcohols, may be in an amount of less than 1 wt. %, e.g., less than 0.5 wt. %, provided that the amount of the other oxygenate compounds is less than the amount of acetic acid. In terms of ranges, the process stream may comprise from 90 wt. % to 99.999 wt. % water and 0.001 wt. % to 10 wt. % acetic acid, e.g., from 95 wt. % to 99.9 wt. % water and 0.1 wt. % to 5 wt. % acetic acid. To avoid a loss of ethanol, the process stream preferably is substantially free of ethanol, e.g., comprises less than 0.5 wt. % ethanol, or less than 0.01 wt. % ethanol.

U.S. Pat. No. 7,829,602 teaches using a pure water stream from a Fischer-Tropsch (FT) when saturating a methane-containing stream. The present invention is also different in that the water is produced by the hydrogenation and in contrast water is fed to the FT process as a heat exchange fluid.

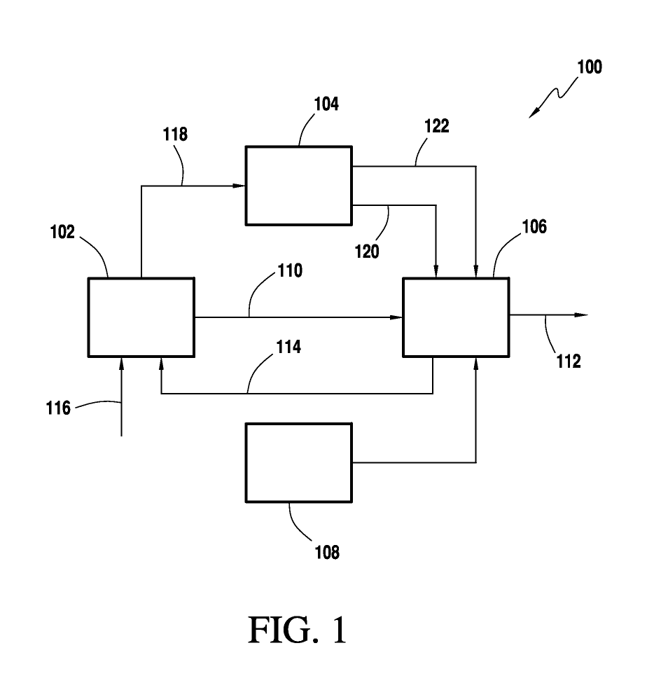

As shown in FIG. 1, an integration system 100 comprises a hydrogen production unit 102, a power generation unit 104, an ethanol production unit 106, and an acetic acid source 108. Acetic acid source 108 may be a storage tank of acetic acid or a co-located acetic acid production unit, such as a carbonylation unit. A pure hydrogen stream 110 produced by hydrogen production unit 102 and acetic acid from acetic acid source 108 are fed to ethanol production unit 106 to produce ethanol 112. In producing ethanol 112, a process stream 114 comprising water and less than or equal to 10 wt. % oxygenates is also obtained. Process stream 114 may be introduced into hydrogen production unit 102 as a source of water to saturate the methane-containing stream 116. Oxygenates in process stream 114 may be converted to syngas in hydrogen production unit 102. Advantageously, the present invention improves efficiencies by integrating process stream 114 with hydrogen production unit 102 to avoid wastewater discharge and to avoid having to solely use an external water stream. Hydrogen production unit 102 also generates a significant amount of high pressure steam 118 that can be converted to electricity 120 and useable low pressure steam 122 in power generation unit 104. Thus, power generation unit 104 may satisfy the electricity and steam needs of ethanol production unit 106.

Hydrogen production unit 102 is simplified to only produce hydrogen as an output. This is advantageous over prior methods, such as those disclosed in EP0167300, which sought to produce various streams of carbon oxides and hydrogen for use in multiple production units, such as methanol production and acetic acid production. This complex integration is avoided by focusing on producing hydrogen without producing streams of carbon oxides and hydrogen. Thus, the present invention is not integrated with methanol production or carbonylation production that requires carbon oxides. Another disadvantage of EP0167300 is that the excess hydrogen produced was used to produce ammonia, which adds to the cost of the integration process.

The equipment associated with each of the process steps according to the present invention may be mounted for portability and/or contained within the footprint of a standard flatbed trailer, barge, towboat, or flat railcar. Reducing the units that are integrated also reduces the footprint of the equipment. In some embodiments, to scale production, the equipment may be contained within multiple footprints of several flatbed trailers, barges, towboats, or flat railcars. For example, hydrogen production unit 102 may be mounted on a trailer and ethanol production unit 106 on a barge. In addition, the present invention may use modular construction to minimize the footprint of the hydrogen and ethanol production units while maintaining ease of operation and maintenance. Lastly, because of the modularity of the production units, the system may be self-sufficient and highly automated, which lends itself to operating in remote locations where stranded natural gas and associated gas are found.

Although the present invention may be advantageously used to improve economic recovery from stranded natural gas by using the hydrogen to produce ethanol, the present invention may also use natural gas from conventional sources. In addition, natural gas may be obtained from landfills or agricultural production. In other embodiments, the methane-containing stream may be obtained from other carbonaceous sources such as coal, petroleum, and/or biomass.

### Hydrogen Production

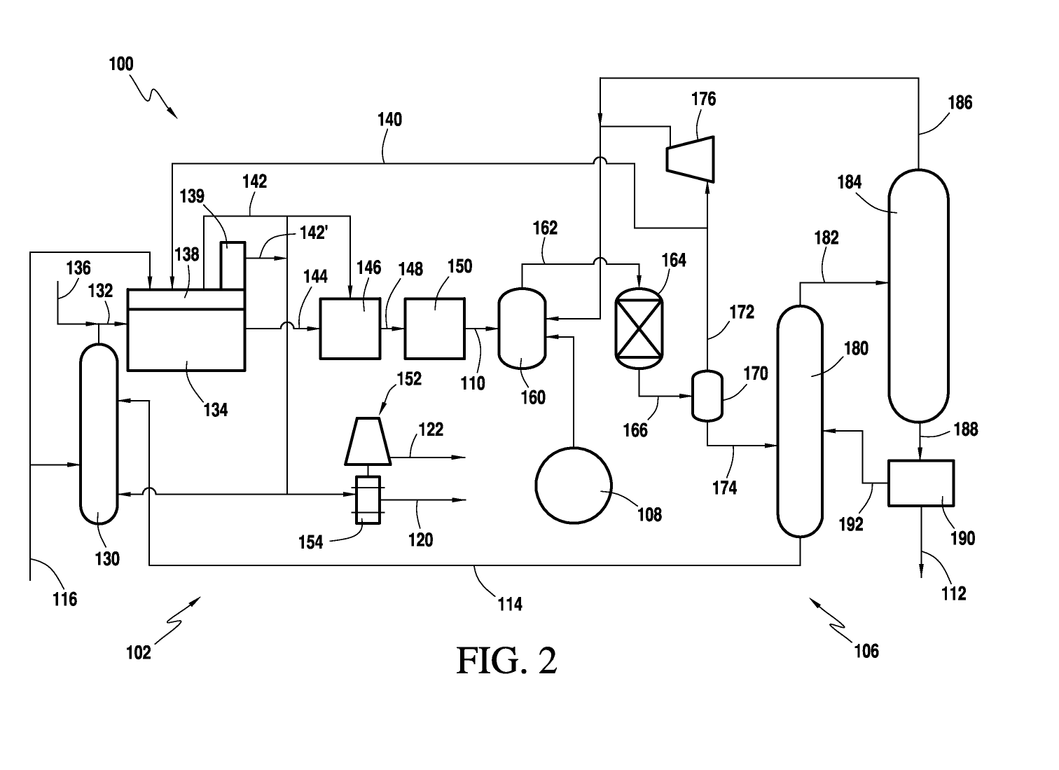

FIG. 2 is a detailed schematic of hydrogen production unit 102 and power generation unit 104. Process stream 114 and methane-containing stream 116 are fed to and are mixed in a saturator 130 to produce a feed stream 132. Saturator 130 may comprise any vessel capable of receiving and mixing methane-containing stream 116 and process stream 114. Saturator 130 provides heat to vaporize the water in the process stream 114. The oxygenates in process stream 114 may also be vaporized. Saturators may include vertical heat exchangers, such falling film evaporators where the methane-containing stream 116 is fed into the bottom and the water, i.e. process stream 114, is fed into the top. Saturators may also include a distillation tower with a thermosiphon reboilers where the water is fed at the top and the methane-containing stream 116 is fed at the bottom. The trays or packing in the tower improve vapor liquid contact between the methane and the water, and optionally oxygenates. No mechanical mixer is needed to saturate methane-containing stream 116. Optionally, additional water that is not obtained from ethanol production unit 106 may be fed to saturator 130 or combined with feed stream 132. Methane-containing stream 116 preferably is obtained from stranded natural gas or associated gas that does not have a uniform composition. The composition of methane-containing stream 116 may vary, and impurities, especially sulfur impurities, may be removed prior to introducing methane-containing stream 116 into hydrogen production unit 102. For example, methane-containing stream 116 may be passed through a catalyst bed to convert organic sulfur-containing compounds into H₂S. The H₂S enriched stream may be passed through absorber, such as a zinc oxide bed, to remove the H₂S.

The purity of methane-containing stream 116 may be dictated by the source and by the economic feasibility of removing impurities. In general, methane-containing stream 116 may have a composition that comprises from 70 mol. % to 99.9 mol. % methane, from 0.1 to 30 mol. % CO₂, from 0.1 to 5 mol. % heavier hydrocarbons (mixtures of ethane, propane, butane, pentane, hexanes, etc. and isomers thereof), and from 0.1 to 1 mol. % nitrogen. Higher amounts of nitrogen, i.e. up to 20 mol. % nitrogen, may be present in some methane-containing stream and a suitable separation of nitrogen and carbon monoxide may be used to reduce the nitrogen concentration. Nitrogen concentrations of up to 5 mol. % may be used with an increase of hydrogen purging. High amounts of CO₂ in the stranded natural gas or associated gas may be tolerated in the present invention and thus the methane-containing stream may comprise from 20 to 30 mol. % CO₂. Thus, pretreatment is not needed to reduce the CO₂.

Process stream 114 may be pre-heated, as needed, to a temperature from 100° C. to 130° C., e.g., from 110° C. to 125° C. or from 115° C. to 120° C. When process stream 114 is not preheated, methane-containing stream 116 may heat process stream 114 in saturator 130. Methane-containing stream 116 may also be pre-heated, as needed, to a temperature that is higher than the temperature of process stream 114. In one embodiment, methane-containing stream 116 may be pre-heated to a temperature from 190° C. to 300° C., e.g., from 200° C. to 275° C. or from 215° C. to 250° C. Saturator 130 may be a pressurized vessel that operates at temperate from 100° C. to 400° C., e.g., from 120° C. to 350° C.

A small blowdown stream (not shown) may be withdrawn from saturator 130 as needed to prevent build up of salts in saturator 130.

Feed stream 132 is withdrawn from saturator 130 and directed to a steam reformer 134. In one embodiment, feed stream 132 may have a molar ratio of steam to methane (water to carbon molar ratio) that is at or slightly below 2.0, e.g., from 1.3 to 2.0 or from 1.5 to 2.0. Low molar ratios are usually avoided to present carbon formation. Feed stream 132 may comprise from 25 to 60 mol. % methane, from 40 to 75 mol. % steam, from 0 to 40 mol. % carbon oxides, from 0 to 5 mol. % hydrocarbons, and from 0 to 5 mol. % oxygenates. The composition of feed stream 132 may vary, but generally it comprises more methane than oxygenates. Makeup steam 136 may be introduced into steam reformer 134 as needed to achieve the desired molar ratio. One or more heat exchangers may be used to preheat feed stream 132 to a temperature from 300° C. to 750° C., e.g., from 400° C. to 500° C. Preferably, feed stream 132 is not condensed.

An exemplary steam reformer 134 may have a multi-tubular in a furnace configuration. Feed stream 132 enters steam reformer 134 and is distributed between a plurality of catalyst-filled tubes. The number of tubes may vary depending on the desired production rates, and may be from 50 to 500 tubes having inner diameters from 0.1 to 30 cm. Any reforming catalyst of appropriate size and shape known to those of skill in the art may be used. In one embodiment, the reforming catalyst may comprise a nickel-based catalyst or a rhodium-based catalyst. Nickel-based catalysts are preferred. Suitable commercial reforming catalysts may include Haldor Topsoe™ RK-201, RK-211, RK-400, and R-67-7H, and Johnson Matthey Katalco 23, 25, and 57 series catalysts. Steam reformer 134 operates at a temperate from 800° C. to 1000° C., e.g., from 850° C. to 950° C., and a pressure from 5 to 40 atm, e.g., from 20 to 35 atm. The heat for steam reformer 134 may be supplied by a convection section, such as a furnace 138. Furnace 138 may be integral with steam reformer 134 and may surround the reactor tubes in the reformer. In one embodiment furnace, 138 is a top fired furnace comprising a convection section and a plurality of tubes. Furnace 138 combines air and natural gas to produce heat. In some embodiments, purge gas 140 from ethanol production unit 106 may be burned in furnace 138. The flue gas leaves furnace 138 at a higher temperature due to the reforming reaction and contains sensible heat to provide steam for export. Preferably, the steam is high pressure steam 142 having a pressure from 40 to 115 atm, e.g., from 50 to 100 atm.

In one embodiment, when additional heat is required, an auxiliary firing unit 139 may be combined with the furnace 138. Thus, the convection section may include both the furnace 138 and auxiliary firing unit 139. Auxiliary firing unit 139 may produce additional high pressure steam 142′ that exceeds the requirements of steam reformer 134. Auxiliary firing unit 139 may be supplied with a combination of purge gas 140 and/or methane-containing stream 116. When integrating with ethanol production unit 106, the heat generating by the auxiliary firing unit 139 may supply nearly all of the steam required to operate the reboilers through low pressure steam 122. Advantageously this improves the integration efficiencies and reduces the footprint of the integrated plant.

Pre-reforming may be used to convert higher hydrocarbons and oxygenates to methane upstream of reforming 134. A suitable pre-reformer may be an adiabatic reactor that contains reforming catalyst. The hydrocarbons and oxygenates may tend to form carbon deposits in the steam methane reactor 134. To avoid such carbon deposits and to increase the conversion of hydrocarbons and oxygenates, a pre-reforming step may be used. Any suitable pre-reforming reactor may be used and the pre-reforming reactor may be operated at a temperature that is less than the reforming step. In one embodiment, the pre-reforming reactor operates at a temperature from 400° C. to 600° C. A nickel catalyst may be used in pre-reforming to convert at least 80% of the hydrocarbons to methane and at least 80% of the oxygenates to methane. The pressure of pre-reforming may be similar to reforming reactor 134.

Reformed effluent 144 is withdrawn from steam reformer 134 and may be cooled using one or more heat exchangers to a temperature from 200° C. to 700° C., e.g., 300° C. to 650° C. Reformed effluent 144 is enriched in hydrogen and carbon monoxide. In one embodiment, reformed effluent 144 comprises from 40 mol. % to 80 mol. % hydrogen, 20 mol. % to 50 mol. % carbon monoxide, and 0 mol. % to 30 mol. % carbon dioxide. Preferably the reformed effluent 144 may comprises from 50 mol. % to 75 mol. % hydrogen, 25 mol. % to 45 mol. % carbon monoxide, and 0 mol. % to 15 mol. % carbon dioxide. The hydrogen to carbon monoxide molar ratio in the reformed effluent 144 may be from 2:1 to 10:1, e.g., from 3:1 to 5:1. Reformed effluent 144 comprises less than 1 mol. % methane and less than 1 mol. % oxygenates. Preferably, all of the methane and oxygenates are converted in steam reformer 134 and thus reformed effluent 144 is substantially free of these compounds.

A shift reaction, i.e. a water gas shift reaction, may be used to further increase the H₂ content and to convert substantially all of the carbon monoxide to carbon dioxide and hydrogen. High pressure steam 142 from furnace 138 may be introduced into a shift reactor 146 along with reformed effluent 144. A shift reaction that converts more than 90% of the carbon monoxide is preferred, e.g., more preferably a reaction that converts more than 95% or more than 99%. An iron-based shift catalyst or copper-based shift catalyst may be used. Exemplary commercial shift catalysts may include Haldor Topsoe™ LK-813 and LK-817 catalysts or Johnson Matthey Katalco™ 71 and 83 series catalysts. The shift reaction may be conducted at a temperate from 175° C. to 500° C., e.g., from 190° C. to 350° C. The shift reaction may be operated at a similar pressure of the reformed effluent 144, thus requiring no further compression downstream of steam reformer 134. In one embodiment, the pressure in the shift reaction is from 5 to 40 atm, e.g., from 10 to 25 atm.

In one optional embodiment, a two stage shift may use two different reactors operating at different temperatures. When a two stage shift is used, it is preferred to use an iron-based catalyst in the first shift reactor at a temperature from 300° C. to 400° C. and a copper-based catalysts in the second shift reactor at a temperature from 190° C. to 210° C.

The product gas 148 of shift reactor 146 may further undergo cooling and dehydration. The cooled product gas 148 is delivered to a carbon dioxide removal unit 150 in which the hydrogen may be separated from the other stream components to produce a pure hydrogen stream 110. Carbon dioxide removal unit 150 may comprise a pressure swing adsorber (PSA), membrane, or an acid gas removal device. An acid gas removal device may use a solvent to remove carbon dioxide such as methanol, dimethyl ether of polyethylene glycol, N-methyl-2-pyrrolidone, N-methyl-diethanolamine, and propylene carbonate. Preferably, at least 95% of the carbon dioxide is removed in the removal unit 150, e.g., at least 99%. The removed carbon dioxide may be sequestered or vented. In one embodiment, hydrogen stream 110 has a purity of greater than 99 mol. % hydrogen, e.g., greater than 99.99 mol. % hydrogen.

### Power Generation

Furnace 138 may generate more steam than can be consumed by the steam reformer. An optional auxiliary firing unit may also be employed to generate excess steam, preferably high pressure steam. The amount of excess steam can be controlled. The excess steam, which is preferably high pressure steam 142 having a pressure from 40 to 115 atm, may be directed to saturator 130 and shift reactor 134 as described above. A portion of the heat pressure steam 142 may be supplied to combined heat and power unit 152. In particular, high pressure stream 142 may be supplied to a topping turbine-generator 154 where it is used to generate electricity 120. The electricity 120 produced is preferably consumed by the ethanol production unit 106 on-site. This may provide small-scale power 1,000 kW to 20,000 kW that is well-suited for use in ethanol production unit 106. The discharge of the topping turbine is low pressure steam 122 having a pressure from 5 to 20 atm, e.g., from 10 to 15 atm. The low pressure steam 122 may be adjusted as needed to be integrated with the heat requirements of ethanol production unit 106. The low pressure steam 122 may be used to pre-heat feeds to the reactor or distillation columns. In addition, the low pressure steam 122 may be used in the distillation column reboilers.

### Ethanol Production

Ethanol production unit 106 produces ethanol by hydrogenating acetic acid or ethyl acetate from source 108. The hydrogen feedstock is supplied as a pure hydrogen stream 110 from hydrogen production unit 102. To integrate ethanol production with stranded natural gas sources, it is preferred that ethanol production unit 106 is co-located with hydrogen production unit 102. Acetic acid or ethyl acetate may also be fed to ethanol production unit 106. In one embodiment, the acetic acid or ethyl acetate production is not located near the stranded natural gas. Acetic acid may be shipped and stored as needed. In addition, by co-locating the hydrogen production unit 102 and ethanol production unit 106, the electricity 120 and low pressure steam 122 produced by hydrogen production unit 102 may be consumed by ethanol production unit 106.

As shown in FIG. 2, pure hydrogen stream 110 and acetic acid or ethyl acetate, or mixtures thereof from source 108 are introduced into a vaporizer 160. In one embodiment, a mixed feed from source 108 may be fed that comprises from 25 wt. % to 95 wt. % acetic acid and from 5 wt. % to 75 wt. % ethyl acetate. Additional recycle streams that comprise hydrogen, acetic acid, and/or ethyl acetate may also be introduced into vaporizer 160. A vapor feed stream 162 is withdrawn and introduced into reactor 164. The temperature of vapor feed stream 162 is preferably from 100° C. to 350° C., e.g., from 120° C. to 310° C. or from 150° C. to 300° C. In addition, although vapor feed stream 162 is shown as being directed to the top of reactor 164, vapor feed stream 162 may be directed to the side, upper portion, or bottom of reactor 164. Any feed that is not vaporized is removed from vaporizer 104, via a blowdown stream.

Reactor 164 contains the catalyst that is used in the hydrogenation of the carboxylic acid, preferably acetic acid. In one embodiment, one or more guard beds (not shown) may be used upstream of the reactor, optionally upstream of vaporizer 160, to protect the catalyst from poisons or undesirable impurities contained in the feed or return/recycle streams. Such guard beds may be employed in the vapor or liquid streams. Suitable guard bed materials may include, for example, carbon, silica, alumina, ceramic, or resins. In one aspect, the guard bed media is functionalized, e.g., silver functionalized, to trap particular species such as sulfur or halogens. During the hydrogenation process, a crude ethanol product 166 is withdrawn, preferably continuously, from reactor 164.

The hydrogenation reaction may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The hydrogenation reactor 164 may be operated at a temperature that is less than the temperature of steam reformer 134. The reactor pressure may range from 100 kPa to 4500 kPa, e.g., from 150 kPa to 3500 kPa, or from 500 kPa to 3000 kPa. The reactants may be fed to the reactor at a gas hourly space velocity (GHSV) from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹. Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 18:1 to 2:1.

Some embodiments of the process of hydrogenating acetic acid to form ethanol may include a variety of configurations using a fixed bed reactor or a fluidized bed reactor. In many embodiments of the present invention, an “adiabatic” reactor can be used; that is, there is little or no need for internal plumbing through the reactor to add or remove heat. In other embodiments, a radial flow reactor or reactors may be employed, or a series of reactors may be employed with or without heat exchange, quenching, or introduction of additional feed material. Alternatively, a shell and tube reactor provided with a heat transfer medium may be used. In many cases, the reactor may be housed in a single vessel or in a series of vessels with heat exchangers therebetween. In addition, reactor may comprise multiple catalyst beds.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles. Contact or residence time can also vary widely, depending upon such variables as amount of acetic acid, catalyst, reactor, temperature, and pressure. Typical contact times range from a fraction of a second to more than several hours when a catalyst system other than a fixed bed is used, with preferred contact times, at least for vapor phase reactions, from 0.1 to 100 seconds.

The hydrogenation of acetic acid to form ethanol is preferably conducted in the presence of a hydrogenation catalyst. Exemplary catalysts are further described in U.S. Pat. Nos. 7,608,744, 7,863,489, 8,080,694, 8,309,772, 8,338,650, 8,450,535, 8,455,702, and 8,471,075, the entireties of which are incorporated herein by reference. In one embodiment, the catalyst comprises two or more metals on a support. The metals may include copper, molybdenum, tin, chromium, iron, cobalt, vanadium, tungsten, palladium, platinum, rhodium, lanthanum, cerium, manganese, gold, nickel, and combinations thereof. Exemplary metal combinations include platinum/tin, platinum/cobalt, platinum/tungsten, platinum/chromium, platinum/palladium, platinum/cerium, palladium/tin, palladium/cobalt, rhodium/tin, cobalt/tungsten, cobalt/chromium, cobalt/zinc, cobalt/tin, copper/palladium, copper/zinc, nickel/palladium, or gold/palladium. In one some embodiments, the hydrogenation catalyst may comprise at least three metals, and includes combinations such as platinum/tin/cobalt, platinum/tin/tungsten, platinum/tin/molybdenum, platinum/tin/copper, platinum/tin/nickel, platinum/tin/chromium, platinum/palladium/cobalt, platinum/palladium/tin, platinum/palladium/copper, rhodium/tin/cobalt, rhodium/tin/tungsten, rhodium/tin/molybdenum, palladium/tin/cobalt, palladium/tin/tungsten, palladium/tin/molybdenum, palladium/tin/copper, palladium/tin/nickel, and palladium/tin/chromium. The total metal loadings may be in an amount from 0.1 to 25 wt. %, e.g., from 0.5 to 15 wt. %, or from 1 to 12 wt. %.

Preferred supports for the hydrogenation catalyst may include silicaceous supports, such as silica, silica/alumina, pyrogenic silica, high purity silica, and mixtures thereof. Other supports may include, but are not limited to, iron oxide, alumina, titania, zirconia, magnesium oxide, carbon, graphite, high surface area graphitized carbon, activated carbons, and mixtures thereof. In some embodiments, the support may be modified with a support modifier as described in U.S. Pat. Nos. 8,080,694, 8,309,772, and 8,471,075, the entireties of which are incorporated herein by reference. The support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIIIB metals, aluminum oxides, and mixtures thereof. Acidic support modifiers include those selected from the group consisting of TiO₂, ZrO₂, Nb₂O₅, Ta₂O₅, Al₂O₃, B₂O₃, P₂O₅, Sb₂O₃, WO₃, MoO₃, Fe₂O₃, Cr₂O₃, V₂O₅, MnO₂, CuO, Co₂O₃, and Bi₂O₃. Preferred support modifiers include oxides of tungsten, molybdenum, and vanadium. In another embodiment, the support modifier may be a basic modifier that has a low volatility or no volatility. Such basic modifiers, for example, may be selected from the group consisting of: (i) alkaline earth metal oxides, (ii) alkali metal oxides, (iii) alkaline earth metal metasilicates, (iv) alkali metal metasilicates, (v) Group JIB metal oxides, (vi) Group JIB metal metasilicates, (vii) Group IIIB metal oxides, (viii) Group IIIB metal metasilicates, and mixtures thereof. The basic support modifier may be selected from the group consisting of oxides and metasilicates of any of sodium, potassium, magnesium, calcium, scandium, yttrium, and zinc, as well as mixtures of any of the foregoing. In one embodiment, the basic support modifier is a calcium silicate, such as calcium metasilicate (CaSiO₃). The calcium metasilicate may be crystalline or amorphous.

In particular, the hydrogenation of acetic acid may achieve favorable conversion of acetic acid and favorable selectivity and productivity to ethanol. For purposes of the present invention, the term “conversion” refers to the amount of acetic acid in the feed that is converted to a compound other than acetic acid. Conversion is expressed as a percentage based on acetic acid in the feed. The conversion may be at least 50%, e.g., at least 75%, or at least 95%. Although catalysts that have high conversions are desirable, such as at least 97% or at least 99%, in some embodiments a low conversion may be acceptable at high selectivity for ethanol. Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, the ethanol selectivity is 60%. Preferably, the catalyst selectivity to ethanol is at least 60%, e.g., at least 70%, or at least 80%. Preferred embodiments of the hydrogenation process also have low selectivity to undesirable products, such as methane, ethane, and carbon dioxide. The selectivity to these undesirable products preferably is less than 4%, e.g., less than 2% or less than 1%.

The term “productivity,” as used herein, refers to the grams of a specified product, e.g., ethanol, formed during the hydrogenation based on the kilograms of catalyst used per hour. The productivity may range from 100 to 3,000 grams of ethanol per kilogram of catalyst per hour.

The composition of crude ethanol product 166 may vary. Crude ethanol product 166 produced by the hydrogenation reaction, before any subsequent processing, such as purification and separation, will typically comprise acetic acid, ethanol and water. Excess hydrogen may also be present. Exemplary compositional ranges for the crude ethanol product are provided in Table 1, excluding hydrogen. The “others” identified in Table 1 may include, for example, esters, ethers, aldehydes, ketones, alkanes, and carbon dioxide.

TABLE 1
CRUDE ETHANOL PRODUCT COMPOSITIONS

| | Conc. | Conc. | Conc. | Conc. |
|---|---|---|---|---|
| Component | (wt. %) | (wt. %) | (wt. %) | (wt. %) |
| Ethanol | 5 to 72 | 15 to 72 | 15 to 70 | 25 to 65 |
| Acetic Acid | 0 to 90 | 0 to 50 | 0 to 35 | 0 to 15 |
| Water | 5 to 40 | 5 to 30 | 10 to 30 | 10 to 26 |
| Ethyl Acetate | 0 to 30 | 1 to 25 | 3 to 20 | 5 to 18 |
| Acetaldehyde | 0 to 10 | 0 to 3 | 0.1 to 3 | 0.2 to 2 |
| Others | 0.1 to 10 | 0.1 to 6 | 0.1 to 4 | — |

At higher conversions, the crude ethanol product may have low concentrations of acetic acid. The crude ethanol product may comprise acetic acid, for example, in an amount ranging from 0.01 wt. % to 20 wt. %, e.g., 0.05 wt. % to 15 wt. %, from 0.1 wt. % to 10 wt. % or from 1 wt. % to 5 wt. %. In embodiments having lower amounts of acetic acid, the conversion of acetic acid is preferably greater than 75%, e.g., greater than 85% or greater than 90%. In addition, the selectivity to ethanol may also be preferably high, and is preferably greater than 75%, e.g., greater than 85% or greater than 90%.

Returning to FIG. 2, crude ethanol product 166 may be condensed and fed to a separator 170, which, in turn, forms a vapor stream 172 and a liquid stream 174. In some embodiments, separator 170 may comprise a flasher or a knockout pot operating at a temperature from 20° C. to 350° C., e.g., from 30° C. to 325° C. or from 60° C. to 250° C. The pressure of separator 170 may be from 100 kPa to 3000 kPa, e.g., from 125 kPa to 2500 kPa or from 150 kPa to 2200 kPa. Optionally, the crude ethanol product 166 may pass through one or more membranes to separate hydrogen and/or other non-condensable gases.

Vapor stream 172 exiting separator 170 may comprise hydrogen, nitrogen, carbon oxides, and light hydrocarbons, and may be returned vaporizer 160. For example, vapor stream 172 may comprise hydrogen, methane, ethane, carbon monoxide, carbon dioxide, nitrogen, and mixtures thereof. Methane may be present in vapor stream 172 in an amount from 0.01 to 3 mol. %. Ethane may be present in vapor stream 172 in an amount from 0.01 to 3 mol. %. Carbon dioxide may be present in vapor stream 172 in an amount from 0.01 to 3 mol. %. In comparison to pure hydrogen stream 110, purged gaseous portion has a low hydrogen concentration.

In some embodiments, the returned vapor stream 172 passes through compressor 176 before being combined with hydrogen stream 110 in vaporizer 160. In one embodiment, a portion of vapor stream 172 is purged as purged gas stream 140, which may have a calorific heating value that may be fed as fuel to furnace 138. This allows the ethanol production unit 106 to remove carbon oxides and light hydrocarbons while recovering the heat value from the purge stream. In addition, using the purge gas stream 140 may minimize hydrocarbon emissions. In one embodiment, less than 15% of vapor stream 172 is purged as purged gas stream 140, e.g., less than 5%. Alternatively, a purge from vapor stream 172 may be flared.

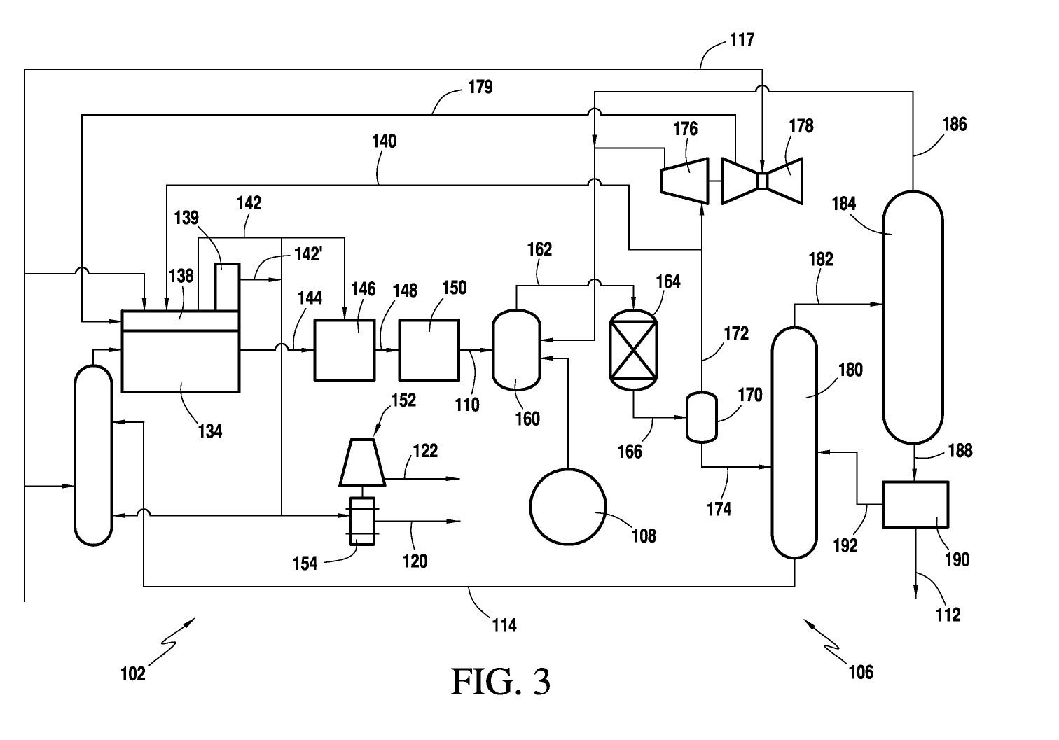

In FIG. 3 there is shown a dedicated turbine 178 for driving compressor 176. A portion of methane-containing stream in line 117 is fed to turbine 178 and the exhaust gases 179 are fed to furnace 138 as a fuel.

To recover ethanol from liquid stream 174, the present invention may employ several different separation techniques and schemes. Exemplary separation techniques and schemes are shown and described in U.S. Pat. Nos. 8,222,466, 8,304,586, 8,304,587, 8,314,272, 8,318,988, 8,440,866, 8,461,399, and US Pub. Nos. 2012/0010438, 2012/0277485, 2012/0273338, 2012/0277490, and 2012/0277497, the entire contents of which are hereby incorporated by reference. The present invention may use any one of these separation schemes. In particular, the separation should achieve recovery of ethanol with low organic impurities and the ability to reduce the water content as needed for the end use, and separation of a process stream that concentrates the water from the hydrogenation reaction. As described above, the process stream preferably concentrates at least 70% of the water from the hydrogenation reaction and contains less than 10 wt. % oxygenates, such as acetic acid. The process stream is integrated by introducing the process stream into a saturator with a methane-containing stream. Advantageously, this eliminates the discharge of water and subsequent remedial cleaning of the water.

FIG. 2 exemplifies one separation and recovery of ethanol from liquid stream 174. Liquid stream 174 is fed to a first column 180, also referred to as an “acid separation column.” First column 180 operates to remove a substantial portion of the water in the residue, which is referred to herein as the process stream 114, depending on the composition of the crude ethanol product, which is a result of the acetic acid conversion and selectivity to ethanol. In one embodiment, 30 to 90% of the water in the crude ethanol product is removed in the residue, e.g., from 40 to 88% of the water or from 50 to 84% of the water. Removing less water in the residue may increase acetic acid carry over in the distillate. In addition, leaving too much water in the residue may also cause increases in ethanol leakage, which is undesirable in the process stream 114. Also, depending on the conversion, the energy requirement may also increase when too much water is left in the first distillate 182. In addition to water, residue may comprise at least 85% of the acetic acid from the crude ethanol product, e.g., at least 90% and more preferably at least about 100%. Preferably, the total amount of acetic acid, based on weight, in residue is less than 10 wt. %. This provides a process stream 114 that can be integrated without having to further separate or remove the acetic acid. This reduces the capital and energy expenditure otherwise needed to purify process stream 114. To achieve integration efficiencies, process stream 114 may be directly fed from first column 180 to saturator 130.

Liquid stream 174 is introduced in the lower part of first column 180, e.g., lower half or lower third. In first column 180, water and acetic acid, along with any other heavy components, if present, are removed from liquid stream 174 and are withdrawn, preferably continuously, as process stream 114. First column 180 also forms an overhead distillate 182, which comprises ethanol and ethyl acetate, and which may be condensed and refluxed, for example, at a ratio of from 10:1 to 1:10, e.g., from 3:1 to 1:3 or from 1:2 to 2:1. In one embodiment, operating with a reflux ratio of less than 5:1 is preferred. When column 180 is operated under about 170 kPa, the temperature of the residue preferably is from 90° C. to 130° C., e.g., from 95° C. to 120° C. or from 100° C. to 115° C. The base of column 180 may be maintained at a relatively low temperature to withdraw a residue stream comprising both water and acetic acid, thereby providing an energy efficiency advantage. The temperature of the first distillate 182 may be from 60° C. to 90° C., e.g., from 65° C. to 85° C. or from 70° C. to 80° C. In some embodiments, the pressure of first column 180 may range from 0.1 kPa to 510 kPa, e.g., from 1 kPa to 475 kPa or from 1 kPa to 375 kPa.

In one embodiment, first column 180 is a tray column having from 5 to 90 theoretical trays, e.g. from 10 to 60 theoretical trays or from 15 to 50 theoretical trays. The number of actual trays for each column may vary depending on the tray efficiency, which is typically from 0.5 to 0.7 depending on the type of tray. The trays may be sieve trays, fixed valve trays, movable valve trays, or any other suitable design known in the art. In other embodiments, a packed column having structured packing or random packing may be employed.

The residue of first column 180 is process stream 114 that is integrated with hydrogen production unit 102. The composition of process stream 114 is provided above.

First distillate 182 that is withdrawn primarily comprises ethanol and ethyl acetate. Other light organics may also be carried over with the ethanol and ethyl acetate. In addition, minor amounts of water may be present in first distillate 182. In one embodiment, the weight ratio of water in the residue to the water in the distillate is greater than 1:1, e.g., greater than 2:1 or greater than 4:1. First distillate 182 preferably is contains less than 600 wppm acetic acid, and more preferably less than 200 wppm acetic acid. This prevents acetic acid may contaminating the ethanol product. In one embodiment, the composition of the first distillate 182 is from 50 to 90 wt. % ethanol, 1 to 40 wt. % ethyl acetate, 4 to 40 wt. % water, 0.01 to 10 vvt. % acetaldehyde, 0.001 to 5 wt. % acetal, and 0.001 to 0.05 wt. % acetone. Depending on reaction conditions, organics such as acetaldehyde, acetal, and acetone may not be present in the crude ethanol product and thus would not build up in first distillate 182. Also, some species, such as acetals, may decompose in first column 180 such that very low amounts, or even no detectable amounts, of acetals remain in the distillate or residue.

Depending on the composition of first distillate 182, one or more columns or separation units may be used to recover an ethanol product having from first distillate 182.

In FIG. 2, first distillate 182 is introduced to a second column 184, also referred to as the “light ends column,” to remove ethyl acetate, and other lights such as acetaldehyde. Ethyl acetate is removed in a second distillate 186 and ethanol is removed as second residue 188. Alternatively, ethanol may also be removed in the lower portion of second column 184 and heavies may be purged in the residue. Second column 184 may be a tray column or packed column. In one embodiment, second column 184 is a tray column having from 5 to 70 theoretical trays, e.g., from 15 to 50 theoretical trays or from 20 to 45 theoretical trays.

To remove ethyl acetate, second column 184 may operate at subatmospheric pressures ranging from 0.1 kPa to 100 kPa, e.g., from 0.1 kPa to 50 kPa or from 0.1 kPa to 35 kPa. Although the temperature of second column 184 may vary, when at about 20 kPa to 70 kPa, the temperature of second residue 188 preferably is from 30° C. to 75° C., e.g., from 35° C. to 70° C. or from 40° C. to 65° C. The temperature of second distillate 186 preferably is from 20° C. to 55° C., e.g., from 25° C. to 50° C. or from 30° C. to 45° C.

In other embodiments, second column 184 may operate at higher pressures and may employ an extractive agent. For the purposes of recovering ethanol any suitable extractive agent may be used, but water is preferred.

The composition of second distillate 186 may comprise from 35 to 90 wt. % ethyl acetate, from 1 to 25 wt. % acetaldehyde, from 0.1 to 10 wt. % water, from 0.01 to 30 wt. % ethanol, and from 0.0001 to 5 wt. % acetal. As shown in FIG. 2, second distillate 186 is returned to vaporizer 160. The hydrogenation catalyst preferably has some activity for converting the ethyl acetate or at least maintaining a steady state of ethyl acetate concentration under steady state conditions. In other embodiments, a portion of second distillate 186 may be returned to hydrogen unit 102 and co-fed with process stream 114 to saturator 130. Depending on the composition, a portion of second distillate 186 may also be purged.

Second residue 188, also referred to as an ethanol enriched stream, preferably comprises from 75 to 99.5 wt. % ethanol, from 0.1 to 25 wt. % water, and less than 0.001 wt. % ethyl acetate. Second residue 188 preferably comprises no acetic acid. Depending on the desired use for ethanol, an ethanol product 112 may be taken directly from second residue 188. More preferably, it is desirable to remove additional water from the second residue 188. Water separator 190 may be an adsorption unit, membrane, molecular sieves, extractive column, or a combination thereof. Most of these water separation techniques require that the feed stream by in vapor phase and optional at a higher pressure. When membranes are used, there may be an array of membranes to remove water. In particular, compression may be necessary when using membranes. Thus, it may be advantageous to withdraw a vapor stream enriched in ethanol from the lower portion of second column 184 and pass the vapor stream to water separator 190. Second residue 188 may be withdrawn as a liquid and vaporized as needed depending on the type of water separator 190.

In one embodiment, the adsorption unit may be a pressure swing adsorption (PSA) unit. The PSA unit may be operated at a temperature from 30° C. to 160° C., e.g., from 80° C. to 140° C., and a pressure of from 0.01 kPa to 550 kPa, e.g., from 1 kPa to 150 kPa. The PSA unit may comprise two to five beds.

A water stream 192 is removed and is preferably returned to first column 180. The water may be fed above the feed point of liquid stream 174 to the first column 180. The water preferably is collected in the residue and directed to hydrogen unit 102 in process stream 114. An ethanol product 112 having a reduced water concentration may be obtained as the desired product from the integrated process.

The associated condensers and liquid separation vessels that may be employed with each of the distillation columns may be of any conventional design and are simplified in the figures. Heat may be supplied to the base of each column or to a circulating bottom stream through a heat exchanger or reboiler. Other types of reboilers, such as internal reboilers, may also be used. The heat that is provided to the reboilers may be derived from any heat generated during the process that is integrated with the reboilers or from an external source such as another heat generating chemical process or a boiler. Although one reactor and one flasher are shown in the figures, additional reactors, flashers, condensers, heating elements, and other components may be used in various embodiments of the present invention. As will be recognized by those skilled in the art, various condensers, pumps, compressors, reboilers, drums, valves, connectors, separation vessels, etc., normally employed in carrying out chemical processes may also be combined and employed in the processes of the present invention.

The temperatures and pressures employed in the columns may vary. Each of the distillation columns may be constructed of a material such as 316L SS, Allot 2205 or Hastelloy C, depending on the operating pressure. Temperatures within the various zones will normally range between the boiling points of the composition removed as the distillate and the composition removed as the residue. As will be recognized by those skilled in the art, the temperature at a given location in an operating distillation column is dependent on the composition of the material at that location and the pressure of column. In addition, feed rates may vary depending on the size of the production process and, if described, may be generically referred to in terms of feed weight ratios.

The ethanol product produced by the processes of the present invention may be an industrial grade ethanol, containing at least 92 wt. % ethanol, or fuel grade ethanol, containing at least 99 wt. % ethanol. The ethyl acetate concentrations for either type of ethanol are less than 100 wppm. In addition, ethanol product typically does not comprise acetic acid. The finished ethanol composition of the present invention preferably contains very low amounts, e.g., less than 0.5 wt. %, of other alcohols, such as methanol, butanol, isobutanol, isoamyl alcohol and other C₄-C₂₀ alcohols. Preferably no methanol is present in the ethanol composition. In one embodiment, the amount of isopropanol in the finished ethanol composition is from 20 to 1,000 wppm, e.g., from 95 to 650 wppm. In one embodiment, the finished ethanol composition is substantially free of acetaldehyde, optionally comprising less than 8 wppm acetaldehyde, e.g., less than 5 wppm or less than 1 wppm.

The finished ethanol composition produced by the embodiments of the present invention may be used in a variety of applications including applications as fuels, solvents, chemical feedstocks, pharmaceutical products, cleansers, sanitizers, hydrogen transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, aldehydes, and higher alcohols, especially butanol. In the production of ethyl acetate, the finished ethanol composition may be esterified with acetic acid. In another application, the finished ethanol composition may be dehydrated to produce ethylene.

While the invention has been described in detail, modifications within the spirit and scope of the invention will be readily apparent to those of skill in the art. In addition, it should be understood that aspects of the invention and portions of various embodiments and various features recited herein and/or in the appended claims may be combined or interchanged either in whole or in part. In the foregoing descriptions of the various embodiments, those embodiments which refer to another embodiment may be appropriately combined with one or more other embodiments, as will be appreciated by one of skill in the art. Furthermore, those of ordinary skill in the art will appreciate that the foregoing description is by way of example only, and is not intended to limit the invention.

What is claimed is:

1. A process for producing ethanol comprising:
hydrogenating acetic acid in a first reactor in the presence of a first catalyst to produce a crude ethanol stream;
separating the crude ethanol stream into an ethanol product stream and a process stream comprising water and oxygenates, wherein the process stream comprises less than or equal to 10 wt. % oxygenates;
mixing the process stream and a methane-containing stream to produce a feed stream;
reforming the feed stream in a second reactor in the presence of a second catalyst to produce a reformed effluent comprising hydrogen, carbon monoxide and carbon dioxide;
shifting substantially of all of the carbon monoxide in the reformed effluent to carbon dioxide and hydrogen; and
removing substantially of all of the carbon dioxide to yield a pure hydrogen stream that is fed to the first reactor.

2. The process of claim 1, wherein the molar ratio of methane to water in the feed stream is at or slightly below 2.0.

3. The process of claim 1, wherein the process stream comprises water and from 0.001 to 10 wt. % oxygenates.

4. The process of claim 1, wherein the process stream is substantially free of ethanol.

5. The process of claim 1, wherein the process stream comprises water and less than 10 wt. % acetic acid.

6. The process of claim 1, wherein the crude ethanol stream comprises water, and further wherein at least 70% of the water in the crude ethanol stream is recovered in the process stream.

7. The process of claim 1, wherein the methane-containing stream comprises 70 mol. % to 99.9 mol. % methane.

8. The process of claim 1, wherein the reforming step produces high pressure steam having a pressure from 40 atm to 115 atm.

9. The process of claim 8, further comprising feeding the high pressure steam to a turbine to generate power, wherein the power is used by the first reactor or by separators for separating the crude ethanol product.

10. The process of claim 9, wherein the turbine discharges low pressure steam, and the low pressure steam is integrated with the separating step for the crude ethanol product.

11. The process of claim 8, wherein the second reactor comprises an auxiliary firing section to generate auxiliary high pressure steam that is fed to the turbine.

12. The process of claim 1, wherein more than 90% of the carbon monoxide is shifted to carbon dioxide.

13. The process of claim 1, wherein the hydrogen to carbon monoxide molar ratio in the reformed effluent is from 2:1 to 5:1.

14. The process of claim 1, wherein the second catalyst is selected from the group consisting of nickel-based catalysts and rhodium-based catalysts.

15. The process of claim 1, wherein the pure hydrogen stream comprises greater than 99.99 wt. % hydrogen.

16. The process of claim 1, wherein the methane-containing stream is obtained from a stranded natural gas source.

17. A process for producing ethanol comprising:
hydrogenating acetic acid in a first reactor in the presence of a catalyst to produce a vapor crude ethanol stream;
condensing the vapor crude ethanol stream to obtain a liquid stream and a hydrogen recycle stream;
purging a gaseous portion of the hydrogen recycle stream;
separating the liquid stream into an ethanol product stream and a process stream comprising water and oxygenates, wherein the process stream comprises less than or equal to 10 wt. % oxygenates;
mixing the process stream and a methane-containing stream to produce a feed stream;
reforming the feed stream in a second reactor in the presence of a second catalyst to yield a pure hydrogen stream that is fed to the first reactor, wherein the second reactor comprises a convection section that comprises a furnace and an auxiliary firing section; and
introducing the purged gaseous portion to the convection section.

18. The process of claim 17, wherein less than 15% of the hydrogen recycle stream is purged into the gaseous portion.

19. The process of claim 17, wherein the purged gaseous portion comprises hydrogen, methane, ethane, carbon monoxide, carbon dioxide, nitrogen, and mixtures thereof.

20. The process of claim 17, wherein the hydrogen concentration of the pure hydrogen stream is greater than the hydrogenation concentration of the purged gaseous portion.
